(19) United States
(12) Patent Application Publication
(10) Pub. No.: US 2005/0118657 A1
(43) Pub. Date: Jun. 2, 2005

(54) Noninvasive screening method for the detection of colorectal cancer

(76) Inventor: Elias J. Corey, Cambridge, MA (US)

Correspondence Address: BANNER & WITCOFF, LTD., 28 STATE STREET, 28th FLOOR, BOSTON, MA, 02109-9601, US

(21) Appl. No.: 10/725,648

(22) Filed: Dec. 2, 2003

(51) Int. Cl. G01N 33/574

(52) U.S. Cl. 435/7.23

(57) ABSTRACT

Disclosed is a method for the detection of colon cancer by a blood test for elevated levels of peptide YY (PYY), a hormone which is produced in the gut, secreted into blood and transported into the brain where it regulates appetite. Other body fluids can also be evaluated in the same way. The use of PYY measurement as part of the periodic medical examination together with other screening tests such as fecal hemoccult and carcinoembryonic antigen could substantially improve the possibility of diagnosis of colon cancer at an early stage when surgery is likely to be curative.

### FIELD OF THE INVENTION

The present invention relates to a simple method for noninvasive diagnosis of colorectal cancer by analysis of a blood sample or other body fluid for the presence of a tumor marker. In preferred embodiments, the marker is the Peptide YY (also known simply as “PYY). A positive test, especially in conjunction with other positive screening tests, identifies candidates for definitive colonoscopy. The objective of the invention is to provide a routine screening tool for the detection of colorectal cancer at a stage sufficiently early to allow curative surgery.

### BACKGROUND OF THE INVENTION

Colorectal cancer (“CRC”) is second as the cause of death among those people in the United States diagnosed with cancer. It is associated with approximately 70,000 deaths, or 14% of all cancer related deaths in men and women. Although colorectal cancer is highly curable by surgery if diagnosed early (Stage I or Stage II at latest), some 50% of patients with this disease die from it.

As used herein, the terms “colon cancer” and “colorectal cancer” are a shorthand expression used to define a number of tumors known to attack the colon and rectum. The World Health Organization (WHO) tumor classification system is the most widely accepted schema for the histologic typing of colorectal tumors, as follows:

1. Epithelial tumors
1.1 Adenoma
   1.1.1 Tubular
   1.1.2 Villous
   1.1.3 Tubulovillous
   1.1.4 Serrated
1.2 Intraepithelial neoplasia (dysplasia) associated with chronic inflammatory diseases
   1.2.1 Low-grade glandular intraepithelial neoplasia
   1.2.2 High-grade glandular intraepithelial neoplasia
1.3. Carcinoma
   1.3.1 Adenocarcinoma
   1.3.2 Mucinous adenocarcinoma
   1.3.3 Signet-ring cell carcinoma
   1.3.4 Small cell carcinoma
   1.3.5 Squamous cell carcinoma
   1.3.6 Adenosquamous carcinoma
   1.3.7 Medullary carcinoma
   1.3.8 Undifferentiated carcinoma
1.4 Carcinoid (well-differentiated endocrine neoplasm)
   1.4.1 EC-cell, serotonin-producing neoplasm
   1.4.2 L-cell, glucagons-like peptide and PP/PYY-producing tumor
   1.4.3 Others
1.5 Mixed carcinoid-adenocarcinoma
1.6 Others

2. Non-epithelial tumors
2.1 Lipoma (benign)
2.2 Leiomyoma (benign)
2.3 Gastrointestinal stromal tumor
2.4 Leiomyosarcoma
2.5 Angiosarcoma
2.6 Kaposi sarcoma
2.7 Malignant melanoma
2.8 Others
2.9 Malignant lymphomas
   2.9.1 Marginal zone B-cell lymphoma of MALT Type
   2.9.2 Mantle cell lymphoma
   2.9.3 Diffuse large B-cell lymphoma
   2.9.4 Burkitt lymphoma
   2.9.5 Burkitt-like/atypical Burkitt-lymphoma
   2.9.6 Others

3. Secondary tumors

4. Polyps
4.1 Hyperplastic (metaplastic)
4.2 Peutz-Jeghers
4.3 Juvenile

Because symptoms of early stage CRC disease are either absent, barely noticeable or non-specific, diagnostic tests are essential for early detection and successful treatment. The main early symptoms, fatigue and weakness, are generally too non-specific to interpret. More meaningful symptoms such as abdominal cramps and changes in bowel habits are also fairly non-specific and occur only when the cancer has become advanced. The most common diagnostic test, fecal occult blood testing for blood in the stool suffers from insensitivity, and high false positive and negative rates. Sigmoidoscopy provides valuable information on the condition of the lower bowel and, if positive for polyps or tumors, can indicate the need for a full colonoscopic examination. A negative result from sigmoidoscopy cannot be interpreted as negative for colon cancer, however. Although colonoscopy is definitive, it is not routinely applied in medicine because of cost ($1000 or greater per procedure), a requirement for rigorous (and onerous) patient preparation, and safety issues (approximately 0.1% risk of complications due to bowel perforation during the procedure). Finally, because colonoscopy requires a skilled gastroenterologist, there are simply not enough practitioners to handle large-scale regular screening of the adult population by this method.

There are a number of potential screening methods which may possibly be of use for the screening of human colon carcinoma using various macromolecular biological markers which are of unproven merit. These methods include the following marker analyses: (1) careinoembryonic antigen (CEA) [(a) Goldman and Friedman, J. Exp. Med., 121, 439-462 (1965), (b) H. Koprowski, Z. Steplewski, M. Herlyn, U.S. Pat. No. 4,471,057, (c) T. R. Barnett, J. J. Elting, M. E. Kamarck, U.S. Pat. No. 5,571,710 and patents cited therein]; (2) hMSH2 protein [A. de la Chapelle, B. Vogelstein, K. W. Kinzler, U.S. Pat. No. 5,837,443]; (3) fecal colonocytes [Po P. Nair, U.S. Pat. No. 6,534,280]. Genetic testing for colorectal cancer predisposition remains on the horizon as a future (perhaps distant) possibility [C. Eng, H. Hempel, A. de la Chapelle, Annu. Rev. Med. 52, 371-400 (2001)].

Because of the enormous human and economic toll of colon cancer, the great benefit that would result from the near term availability of a practical and simple noninvasive diagnostic test and the absence of such effective screening methods in current medical practice, provided the motivation to perform the studies leading to the invention set forth below. The need for such an invention is clear from recent reviews from the medical literature, including: (1) “Colorectal Cancer: The Importance of Early Detection,” At-A-Glance (1996); (2) K. Isselbacher, E., Braunwald, et al., Harrison's Principles of Internal Medicine, 257, 1424 (1994); (3) N. W. Toribara and M. H. Sleisenger, “Screening for Colorectal Cancer.” N. Engl. J. Med. 332, 861 (1995). (4) D. E. Mansell, “Colon Polyps and Colon Cancer,” http://personalweb.sunset.net/mansell/polyp.htm (5) H. Cui, M. Cruz-Correa, et al. “Loss of IGF2 Imprinting: A Potential Marker of Colorectal Cancer Risk, Science, 299, 1753 (2003). (6) W. S. Samowitz, et al. Gasteroenterology, 121, 830 (2001). (7) M. J. Khoury, L. L. McCabe, E. R. B. McCabe, “Population Screening in the Age of Genomic Medicine,” N. Eng. J. Med., 348, 50 (2003). (8) U. Kressner, G. Lindmark et al., “Heterogeneity of Proliferation Markers in Colorectal Cancer,” Anticancer Res. 15, 2755 (1995). (9) C. D. Johnson et al., “Computerized Tomographic Colonography: “Performance Evaluation in a Retrospective Multicenter Setting,” Gastroenterology, 125, 688 (2003). (10) D. F. Ransohoff, C. A. Lang, “Screening for Colorectal Cancer,” N. Engl. J. Med., 325, 37 (1991). (11) P. A. Janne, Robert 1. Mayer, “Screening for Colorectal Cancer,” N. Engl. J. Med., 342, 1960 (2000). (12) G. N. Ioannu, M. K. Chapko, J. A. Dominitz, “Predictors of Colorectal Cancer Screening Participation in the United States,” Am. J. Gasteroent., 98, 2082 (2003).

### SUMMARY OF THE INVENTION

The present invention is based upon the discovery that colon tumor cells (see tumor listing above) overproduce the 36 amino acid peptide PYY, and secrete it into the blood and surrounding body fluids. A baseline level of PYY for a mammal not suffering from colon cancer may be established by routine blood testing. Thereafter, any elevation of PYY levels in blood—above this baseline—serve as an indicator that further studies should be made as to the health of the patient's colon.

Thus, one embodiment of the invention is a colon cancer screening test comprising the steps of:
(a) establishing a baseline level of Peptide YY in a body fluid sample from a mammalian patient; and
(b) comparing the level of Peptide YY in a subsequent sample of the patient's body fluid with a the baseline level subsequent body fluid; and
(c) wherein an elevated level of Peptide YY in the subsequent sample indicates a positive result for colon cancer.

Preferably, steps (a) and (b) are conducted on a routine time basis, as selected by the physician and/or the patient. Typical time periods for such cancer screening tests include monthly, quarterly, semi-annually and annually—depending upon the degree of change of the patient's PYY levels—if any.

PYY is a hormonal peptide of 36 amino acids with the sequence:
YPAKPEAPGE¹⁰DASPEELNRY²⁰ YASLRHYLNL³⁰ VTRQRY (NH₂)³⁶
   wherein the symbols used for this structure are the standard one-letter amino acid designations. PYY is derived from a precursor protein of 97 amino acids by selective endopeptidase cleavage of the amide linkages to Y²⁹. The C-terminal Y of PYY is in the amide form.

### DETAILED DESCRIPTION OF THE INVENTION

The nature, function and analytical methodology of PYY are discussed below. Next, evidence is given for the utility of PYY levels in blood and other appropriate body fluids, for example fecal, as a marker for colon cancer. Then, protocols are outlined for the clinical use of PYY measurements in routine, large-scale screening for colon cancer. Test kits for establishing baseline measurements and subsequent monitoring measurements can be prepared for PYY as with other markers for cancer—such as the well-known PSA test kits available from numerous commercial sources.

Function, formation and secretion of PYY by the colon:

Both PYY and the precursor protein are synthesized in the gut (small bowl and colon) and secreted therefrom. The synthesis of PYY and its secretion into the blood are stimulated after food intake by the arrival of partially digested food (especially fats and bile acids) into the gut. The PYY so produced is transported via the blood into the brain where it produces a satiety signal that minimizes any desire for additional food. PYY infusion into blood reduces hunger both in lean and obese humans.

Natural PYY levels tend to be lower in obese than in lean people, indicating that PYY deficiency may plan a role in obesity; see: (1) R. L. Batterham, M. A. Cowley et al., “Gut Hormone PYY (3-36) Physiologically Inhibits Food Intake,” Nature, 418, 650 (2002). (2) R. L. Batterham, S. M. Ellis, C. W. Le Roux, et al. in N. Engl. J. Med. 349, 941 (2003) and related comments by J. Komer and R. L. Liebel in N. Eng. J. Med. 349, 926 (2003). PYY thus has the opposite action as compared to the orexigenic 28 amino acid peptide ghrelin which is made in the stomach and secreted into blood to produce premeal hunger through its actions on the brain. [For information on ghrelin see: (1) M. Kojima, H. Hosoda, Y. Date, et al., Nature, 402, 656 (1999). (2) A. M. Wren, L. J. Seal, M. A. Cohen et al., “Ghrelin enhances appetite and increases food intake in humans,” J. Clin. Endocrinol. Metab. 86, 5992 (2001). (3) D. E. Cummings, D. S. Weigle, R. S. Frayo, et al., “Plasma ghrelin levels after diet-induced weight loss or gastric bypass surgery,” N. Engl. J. Med. 346, 1623 (2002).] There is much current interest in PYY and ghrelin in connection with the understanding and control of obesity.

The three-dimensional shape of PYY has been determined by nuclear magnetic resonance spectroscopy [D. A. Keire, M. Kobayashi, T. E. Solomon, J. R. Reeve, Jr., “Solution Structure of Monomeric Peptide YY Supports the Functional Significance of the PP-Fold,” Biochem., 39, 9935 (2000)]. PYY exerts its hormonal satiety effect in the brain mainly by binding to the Y2 receptor, a G-protein-coupled receptor [R. L. Batterham, S. R. Bloom, “The Gut Hormone PYY Regulates Appetite,” Ann. N. Y. Acad. Sci., 994, 162 (2003).] A phosphorylated form of PYY, Ser 13 phospho PYY, in the gut, also binds to the Y2 receptor [Z.-w. Chen, E. Eriste, A. P. Jonsson, et al., FEBS Lett. 492, 119 (2001)].

PYY levels in body fluids, such as, for example in blood, can be measured by a variety of standard analytical methods for peptides. These methods include radioimmunoassay (RIA), enzyme linked immunoassay (ELISA), and automated high performance liquid chromatographic analysis, capillary electrophoresis, and mass spectrometry [see, B. M. Dunn, “Peptide Analysis Protocols,” BACHEM Bioscience]. RIA and ELISA analysis kits are sold commercially, for example by Phoenix Pharmaceuticals, Inc. (www.phoenixpeptide.com). Antibodies to PYY are commercially available, for example from Alpha Diagnostic International, Inc., 5415 Lost Lane, San Antonio, Tex. 78238 (www.4adi.com).

Upregulation of PYY in Colon Cancer:

Tumor cells proliferate uncontrollably and at rates much greater than normal cells. Correspondingly their metabolism and biochemical activity is greatly enhanced, as was demonstrated many years ago by Otto Warburg whose classic research uncovered the much greater uptake of oxygen by tumor cells as compared to their normal counterparts. It is logical, therefore, that the production and secretion of PYY would be enhanced in cancerous colon cells, especially since PYY stimulates proliferation of gastrointestinal mucosa [H.-M. Lee, V. Udupi, E. W. Englander, et al., Endocrinology, 140, 4065 (1999)].

General upregulation of cell metabolism, typical of tumor cells, in itself could enhance PYY production, regardless of any proliferative effect. However, the most important connection between colon cancer and PYY overproduction comes from the observation that weight loss frequently occurs during the progression of colon cancer, commencing even in advance of the earliest symptoms. Weight loss is seldom appreciated by patients, who may in many cases be motivated to lose weight and see minor weight loss as highly beneficial. Older individuals who have a tendency to gain weight readily are generally pleased to experience a diminished tendency to add weight. It is invariably only after the diagnosis of colorectal cancer that weight loss is realized as the earliest marker for the illness by either patient or physician.

Research conducted in this investigation shows that facile weight stabilization or loss generally sets in before the progression of colon cancer has advanced to the incurable stage, most likely at a time corresponding to Stage I or early Stage II colon cancer when surgery should be curative. The present invention is thus based on the connection between the loss of weight due to increased PYY production in the gut—thus leading to the concept that an increase in blood levels of PYY could be detected by routine annual or semiannual testing, for instance by analysis of a blood sample for PYY under carefully standardized conditions. The most favorable conditions for such a blood test are likely to be after overnight fasting to eliminate variability due to food intake and to enable comparison with a baseline value for each patient.

The detection of elevated PYY levels coupled with a positive repeat fecal hemoccult test would be a strong indicator of the possibility of colon cancer and the desirability of additional diagnostic procedures, including testing for other cancer markers (such as CEA) and/or colonoscopy. It is unlikely that minor weight loss, per se, would be of any diagnostic value, and in any event is only noticed retrospectively.

Changes in PYY levels will also correlate with the stages of disease and the responses to various treatments in patients. Therefore sequential measurements of PYY levels will be an important tool in monitoring patients with CRC and in determining the effectiveness of any non-surgical therapies.

It is dogma among physicians that cachexia (weight loss and wasting syndrome) is generally associated with cancer, and increasingly so as the disease advances. The conventional wisdom is that because cancer patients experience weakness, fatigue and pain, they naturally lose appetite and eat less and that this is one cause of weight loss. In biochemical terms, increased production of tumor necrosis factor (TNF-alpha), a consequence of diseases such as cancer (or AIDS), sets in motion a biochemical cascade that leads to anorexia and catabolism of protein and lipid. [(a) M. Buck, L. Zhang, N. A. Halaz, et al., “Nuclear export of phosphorylated C/EPBbeta mediates the inhibition of albumin expression by TNF-alpha,” EMBO Journal, 20, 6712 (2001). (b) K. J. Tracey and A. Cerami, “Tumor necrosis factor and regulation of metabolism . . . ” Proc. Soc. Exp. Biol., 200, 233 (1992).] Cachexia is a major factor in cancer-related mortality in approximately one-fifth of cancer deaths. Thus, it is easy to understand why physicians that were confidentially interviewed were surprised that weight loss provided an important clue to this invention.

In summary, this invention links the overproduction of the gut-brain hormone PYY to the presence of a colon tumor. Fortunately, the level of PYY in body fluids such as blood is readily measured and can serve as a practical and economical screening test for colon cancer at a stage when surgery is likely to be curative.

It is possible that other gut-brain hormones may also be overexpressed and over secreted by colon tumor cells. It is reasonable that profiling of the levels of these peptides, for example in blood, may provide additional diagnostic information. These peptides include somatostatin, neurotensin and the enteroglucagons.

1. A colon cancer screening test comprising the steps of:
(a) establishing a baseline level of Peptide YY in a body fluid sample from a mammalian patient; and
(b) comparing the level of Peptide YY in a subsequent sample of the patient's body fluid with the baseline level as measured for the same body fluid; and
(c) wherein an elevated level of Peptide YY in the subsequent sample indicates a positive result for colon cancer.

2. The colon cancer screening test of claim 1, wherein step (a) is preceded by overnight fasting to eliminate variability due to food intake.

3. The colon cancer screening test of claim 1, wherein step (b) is preceded by overnight fasting to eliminate variability due to food intake.

4. The colon cancer screening test of claim 1, wherein step (a) is repeated on a time period selected from the group consisting of monthly, quarterly, semi-annually and annually.

5. The colon cancer screening test of claim 1, wherein step (b) is repeated on a time period selected from the group consisting of monthly, quarterly, semi-annually and annually.

6. The colon cancer screening test of claim 1, wherein the body fluid sample is blood.

7. The colon cancer screening test of claim 1, wherein the body fluid sample is plasma.

8. The colon cancer screening test of claim 1, wherein the body fluid sample is intestinal or fecal fluid.

9. The colon cancer screening test of claim 1, wherein the PYY levels are measured by a standard protocol for peptide analysis.

10. The colon cancer screening test of claim 9, wherein the standard protocol for peptide analysis is selected from the group consisting of radioimmunoassay (RIA), enzyme linked immunoassay (ELISA), and automated high performance liquid chromatographic analysis, other analytical chromatographic protocols, capillary electrophoresis, and mass spectrometry.
